SEQUENCE LISTING (3 sequences)

SEQ ID NO: 1 - DNA, 20 bp, Artificial Sequence
  Blon_2348 sialidase tgene primer Inf2348F
atacagcagaaccttggcct

SEQ ID NO: 2 - DNA, 20 bp, Artificial Sequence
  Blon_2348 sialidase tgene primer Inf2348R
gcgatcacatggacgagaac

SEQ ID NO: 3 - DNA, 24 bp, Artificial Sequence
  Blon_2348 sialidase tgene primer Inf2348_P
  6-FAM, 6-carboxyfluorescein (1)..(1)
  ZEN quencher (9)..(9)
  3IABkFQ, Iowa Black fluorophore quencher (24)..(24)
tttcacggatcaccggaccatacg

(19) United States
(12) Patent Application Publication
(10) Pub. No.: US 2024/0024386 A1
(43) Pub. Date: Jan. 25, 2024

(54) MICROBIOME MEDIATED INDUCTION OF IMMUNE TOLERANCE AND RESOLUTION OF INFLAMMATION

(71) Applicant: INFINANT HEALTH, INC., Davis, CA (US)

(72) Inventors: David KYLE, Davis, CA (US); Bethany HENRICK, Davis, CA (US)

(73) Assignee: INFINANT HEALTH, INC., Davis, CA (US)

(21) Appl. No.: 18/018,178

(22) PCT Filed: Jul. 27, 2021
(86) PCT No.: PCT/US2021/043339

§ 371 Date: Jan. 26, 2023

Related U.S. Application Data
(60) Provisional application No. 63/057,262, filed on Jul. 27, 2020.

(51) Int. Cl. A61K 35/747; A61K 45/06; A61K 35/745; A61K 47/14; A61K 31/702; A61K 31/202; A61K 31/07; A61K 31/375; A61P 37/04; A61K 47/44; A23L 33/12; A23L 33/155; A23L 33/135; A23L 33/00; A23L 33/125

(52) U.S. Cl. CPC A61K 35/747; A61K 45/06; A61K 35/745; A61K 47/14; A61K 31/702; A61K 31/202; A61K 31/07; A61K 31/375; A61P 37/04; A61K 47/44; A23L 33/12; A23L 33/155; A23L 33/135; A23L 33/40; A23L 33/125

(57) ABSTRACT

The invention described herein relates generally to methods and compositions for administration to modulate the microbiome for the express purpose of increasing host interferon beta (IPNβ) in mammals of target populations that are at risk for or are being treated for certain inflammatory conditions including infections, viral infection, autoimmune conditions, and recovery from sequalae associated with acute inflammatory insults. In particular, the invention provides a method of elevating the level of IPNβ in the gut of a mammal by providing the mammal with a live bacterium and a prebiotic that the live bacterium is known to consume. Further, the invention described herein may relate to the administration of compositions found to increase the endogenous production of IPNβ in the target mammal.

### CROSS-REFERENCE TO RELATED APPLICATIONS

This application is a 35 U.S.C. § 371 National Phase Entry Application of International Patent Application No. PCT/US2021/043339 filed on Jul. 27, 2021, which designated the U.S., and which claims benefit under 35 U.S.C. § 119(e) of U.S. Provisional Application No. 63/057,262 filed Jul. 27, 2021, the contents of which are incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on Jan. 25, 2023, is named 096910-192850USPX_SL.txt and is 1,166 bytes in size.

### FIELD OF INVENTION

The invention described herein relates generally to methods and compositions for administration to modulate the microbiome for the express purpose of increasing host interferon beta (IFNβ) in mammals of target populations that are at risk for or are being treated for certain inflammatory conditions including autoimmune conditions and recovery from sequalae associated with acute inflammatory insults. Further, the invention described herein may relate to the administration of compositions found to increase the endogenous production of IFNβ in the target mammal.

### BACKGROUND

IFNβ is known to inhibit production of the inflammatory Th1 cytokines, IFNγ and interleukin 12 (IL-12), by human dendritic cells. Furthermore, long-term use of IFNβ suppresses the expression of Th1/Th2/Th17 inflammatory cytokines. Presence of IFNβ decreases activity of certain T cells, thereby reducing endogenous production of associated cytokines in the infant gut which are thought to play a role in the development of allergic and autoimmune disease. IFNβ is a naturally produced cytokine known to reduce inflammatory processes and is anti-viral. IFNβ is presently used in the treatment of multiple sclerosis via intramuscular injection.

The gut microbiome is a community of microorganisms residing in the gut; it has been shown to be critical for the organism's healthy early-life development. In particular, a healthy composition of the gut microbiome has been demonstrated to be crucial in the development of a healthy immune system. Disruption of immune homeostasis early in life can lead to increased risk of the onset and progression of various autoimmune and allergic diseases. Recently, a longitudinal study of newborn human infants using systems-level immune analyses found that gut dysbiosis in infants is associated with increased circulating endothelial cells, activated effector T cells, and inflammatory cytokine production.

The role of IFNβ as a central signaling mechanism in maintaining intestinal homeostasis and preventing inflammatory and autoimmune diseases of adults, such as celiac disease, psoriasis, multiple sclerosis, and cancer, has been well established (Rackov, 2017). However, little is known about the ability of the gut microbiome to alter the production and role of IFNβ in immune system development in human infants and recovery from an acute infection or the resolution of chronic inflammation.

Recombinant IFNβ has been used to treat certain disease conditions like Multiple Sclerosis. Recombinant IFNβ has been shown to interfere with viral replication. Nebulized IFNβ alpha 2b has been shown to reduce duration of detectable COVID-19 in humans (doi: 10.3389/fimmu.2020.01061). IFNβ 1a inhibits SRS-CoV in Vero E6 cells (Hensley USAMRID; 2004). A triple therapy of IFNβ-1b using lopinavir—ritonavir, and ribavirin shortened the period of viral shedding for COVID-19 (Lancet 2020; 395: 1695-704).

### SUMMARY OF INVENTION

Methods and compositions described herein comprise elevating the level of interferon beta (IFNβ) in a mammal who has or is at risk of an infection or disease, such compositions and methods comprise live bacterium and one or more mammalian milk oligosaccharides (MMO) consumed by that live bacterium. The IFNβ may be elevated to a therapeutically effective level for the prevention or treatment of an acute or chronic viral infection, an autoimmune or allergic disease. The live bacterium may be selected from Bifidobacterium or Lactobacillus. Bifidobacterium may be selected from, but not limited to, the following species: B. adolescentis, B. animalis, B. animalis subsp. animalis, B. animalis subsp. lactis, B. bifidum, B. breve, B. catenulatum, B. longum, B. longum subsp. infantis (B. infantis), B. longum subsp. longum (B. longum), B. longum subsp. suis, B. pseudocatanulatum, and B. pseudolongum. A preferred Bifidobacterium is B. infantis. Optionally, the B. infantis is activated. Preferably, the B. infantis contains a functional H5 gene cluster. For example, the B. infantis may be strain EVC001 or a genetic equivalent. The Lactobacillus may be selected from the group consisting of L. acidophilus, L. brevis, L. casei, L. crispatus, L. curvatus, L. fermentum, L. pentosus, L. plantarum, L. sakei, L. antri, L. coleohominis, L. gasseri, L. johnsonii, L. mucosae, L. reuteri, L. rhamnosus, and L. salivarius. A preferred Lactobacillus for this invention may be L. rhamnosus or the LGG strain or an L. reuteri.

The composition may comprise live bacterium in an amount of 0.1 million-500 billion Colony Forming Units (CFU) per gram, per serving, or per unit dose delivered as part of a composition. The composition may contain the live bacterium in an amount of 0.001-100 billion Colony Forming Units CFU, 0.1 million to 100 million, 1 million to 5 billion, or 5-20 billion CFU per gram, per serving, or per unit dose. The live bacteria may be in an amount of 0.01, 0.1, 1, 5, 15, 20, 25, 30, 35, 40, 45, or 50 billion CFU per gram, per serving or per unit dose delivered as part of a composition. The live bacterium may be in an amount of 5-20 billion CFU per gram, per serving size or per unit dose of the composition or 5-20 billion CFU per gram of composition or 0.1 million to 100 million CFU per gram of composition.

MMO may include one or more of lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof.

In some embodiments, the HMO is Lacto-N-tetraose (LNT) or Lacto-N-neotetraose (LNnT). In other embodiments, the HMO contains LNT and LNnT. In some embodiments, the LNT and LNnT are in a 10:1, 4:3, 5:1, 2:1, 1:1 1:2, 3:4 or 1:10 ratio, or a range thereof. In some embodiments, the HMO contains at least one sialic acid containing oligosaccharide structure that is not sialyl lactose, at least one fucose containing oligosaccharide that is not fucosyl lactose and either LNT or LNnT. In yet other embodiments, the MMO has at least 2 MMO selected from 2′FL 3′FL, 3′SL, 6′SL LNT, LNnT, LNFP, and DFL.

In some embodiments, the composition further comprises GOS, PDX, FOS or other resistant starch.

MMO may be administered in a composition such that the mammal receives 1-50 grams of MMO per day. MMO may be administered as a concentration, such that the mammal receives a certain amount per serving or at one time. Compositions may comprise 4 gram/liter (g/L), at least 8 g/L, at least 12 g/L, at least 15 g/L. at least 20 g/L of one or more oligosaccharides chosen to promote the growth of one or more commensal organisms, including sources that are consumed by the live bacterium administered. In some embodiments, MMO consumed reach a daily intake of at least 1 gram (g), at least 4 g, at least 8 g, at least 12 g, at least 15 g, at least 20 g, at least 25 g, at least 30 g, at least 35 g, at least 40 g, at least 45 g, at least 50 g, or more than 50 g.

In some methods, compositions further comprise other nutritive components that when administered to a mammal facilitate the production of IFNβ. Nutritive components include but are not limited to oils such as medium chain triglyceride (MCT), palm, soy, high oleic sunflower oil, or coconut. Minerals, certain types of proteins including glycosylated whey protein, tryptophan and other amino acids, soy lecithin, milk fat globule membrane complexes.

This invention also contemplates a method of reducing viral carriage in a mammal by providing said mammal with a composition comprising a live bacterium and a prebiotic or MMO that is consumed by that live bacterium. In some embodiments, it is an antiviral medicament for a mammal comprising a bacterium and a prebiotic. This invention also contemplates a method for reducing the severity of the infection and/or comorbidities of a human with a Corona Virus by providing the individual with from 10⁶ to 10¹¹ CFU of B. infantis contemporaneously with from 1-50 g of a mammalian milk oligosaccharide; and optionally repeating the daily dosing through the time of a viral outbreak. This invention also contemplates a method for preventing the infection of a human who may have been in contact with another person who was carrying a Corona Virus by providing the human with from 10⁶ to 10¹¹ cfu of B. infantis contemporaneously with from 1-50 g of a mammalian milk oligosaccharide; monitoring the human for the presence of a Corona Virus; and repeating the daily dosing for from 14-21 days.

This invention also contemplates a method of reducing gut dysbiosis-associated autoimmune and/or allergic disease by providing a mammal with a composition comprising a live bacterium and a prebiotic or MMO that is consumed by that live bacterium. Gut dysbiosis-associated autoimmune and allergic disease refers to any allergy or autoimmune disorder that can be reasonably traced to gut dysbiosis. Such diseases may include, but are not limited to, rheumatoid arthritis, psoriasis, celiac disease, inflammatory bowel diseases (Crohn's, ulcerative colitis,) IBS, Type 1 diabetes mellitus, atopic dermatitis, asthma, and food allergies or development of oral tolerance to known common food allergens, including specific allergenic proteins from milk, egg, soy, shellfish, fish, tree nuts, peanuts, or sesame.

A chronic viral infection may be HIV, herpes, or hepatitis. An acute infection may be from the family Coronaviridae, influenza, respiratory syncytial virus, among others.

A therapeutically effective dose is any dosage of IFNβ which has the effect of preventing or reducing acute symptoms caused by a gut dysbiosis associated autoimmune or allergic disease or a viral infection such as COVID. A therapeutically effective dose of IFNβ may include in part exogenously added IFN-beta and/or IFN-beta produced endogenously in response to the administration of bacteria and MMO according to this invention.

A mammal may be a human, a non-human primate, a cow, sheep, goat, horse, buffalo, pig, dog or cat.

Any of the compositions of this invention may be tailored or targeted to specific age groups, such as a preterm infant who may be born with a gestational age of less than 33 weeks, the preterm babies may be an extremely low weight (ELBW), very low birth weight (VLBW), or low birth weight (LBW), a term infant (0-3 months), an infant 3-6 months, an infant (6-12 months), a weaning infant (4-12 months), a weaned infant (12 months to 2 years) and toddler (1-3 years), child (3-16 years), an adult (16-70 yr), or an older or geriatric adult (70-100+ yr).

The mammal treated by the method of this invention may be hospitalized or in an outpatient treatment.

### DESCRIPTION OF FIGURES

FIG. 1 Correlations between specific gut taxa and enteric inflammatory cytokine production. A. Heatmap shows correlations between bacterial families and specific cytokines computed via Spearman correlation. P values are corrected using Benjamini-Hochberg procedure (false discovery rate) to estimate significant correlations between microbial taxonomic compositions and specific cytokine concentration detected in the feces of exclusively breastfed infants at day 60 postnatal. B. Total Bifidobacterium abundance was correlated with IFNβ concentration at day 60 postnatal. C. Total Clostridiaceae abundance was correlated with IFNβ concentration at day 60 postnatal. D. Total Enterobacteriaceae abundance was correlated with IFNβ concentration at day 60 postnatal. Each cytokine was tested in duplicate three different times. P values were adjusted and considered to be statistically significant is *P<0.05, **P<0.01,*** P<0.001.

FIG. 2 Fecal IFNβ levels are dependent on the abundance of Bifidobacteriaceae. A. Forty fecal samples from day 60 were evaluated for IFNβ concentration and compared to relative abundance of Bifidobacteriaecae species at day 21. Total IFNβ concentrations were evaluated in fecal samples that contained no Bifidobacteriaceae.

FIG. 3. Specific species of Bifidobacterium were analyzed and only B. longum (that includes B. infantis sub sp.) relative abundance was directly correlated with IFNβ concentration (R=0.48, p=0.0028).

FIG. 4. Infants fed B. infantis EVC001 had significantly modulated fecal cytokine levels compared to controls A. Radar plot representations of median cytokine concentrations [pg/mg] detected in fecal samples from the controls (n=20) and infants fed B. infantis EVC001 (n=20; EVC001) on day 6 (baseline). B. Control infants at day 60 postnatal. C. EVC001 fed infants at day 60 postnatal. Median values were adjusted to log scale, then normalized within each cytokine from 0 to 1. Statistical analysis was completed between controls and EVC001-fed infant fecal samples at day 60 postnatal using Wilcoxon rank-sum test. P values were adjusted using Bonferonni-Holm method and considered statistically significant if *P<0.05, **P<0.01, *** P<0.001.

FIG. 5. increase in IFNβ and decrease in other inflammatory markers correlate with improved growth. Weight for age (WAZ), weight for length WLZ or overall weight gain.

### DETAILED DESCRIPTION OF INVENTION

Inventors disclose herein the discovery of novel methods found to increase the presence of IFNβ in the mammalian gut, thereby reducing the risk of these mammals developing autoimmune and allergic diseases at critical periods of immune development and helping resolve inflammation that results from viral infection. The invention herein relates to triggering endogenous interferon β (IFNβ) production to protect human tissues. The inventors unexpectedly found that fecal IFNβ was elevated in newborn breastfed infants when they were fed B. infantis. IFNβ was elevated when B. infantis remodeled the microbiome composition and changed the biochemistry of the gut and altered the fecal inflammatory profile. The invention relates to modulating the human gut microbiome through specific methods and compositions that generally lead to increased production or presence of IFNβ. Furthermore, the induction of endogenous production, or the administration of IFNβ may curb endogenous production of other cytokines which are associated with allergic and autoimmune disease.

Such methods and compositions may be used to prevent autoimmune and allergic diseases or treat existing diseases or symptoms associated with a disorder. Such disorders may include, but are not limited to rheumatoid arthritis, multiple sclerosis, celiac disease, inflammatory bowel diseases (Crohn's, ulcerative colitis,) IBS, Type 1 diabetes mellitus, psoriasis, atopic dermatitis, asthma, and food allergies. Such methods and compositions may also be used for target populations which are particularly at risk of developing diseases due, for example, to family history and/or the disruption of the infant gut microbiome early in life.

This same mechanism could be used to resolve viral mediated inflammation in the post-acute phase of an illness such as viral infections where neuronal and other systemic damage may contribute to prolonged hospitalization and morbidity. Viruses may include RNA viruses from the Coronaviridae family, such as the virus which causes COVID19. This treatment may be used in humans of all ages by providing combinations of B. infantis and certain oligosaccharides to the patient. The patient may be hospitalized or an outpatient receiving following-up treatment and monitoring. In some embodiments, success of treatment may result in less viral shedding, viral carriage, complications (including neuronal complications), or improved recovery time. Stimulating endogenous IFNβ may also be used in the management of chronic viral infections such as HIV, Hepatitis and Herpes.

IFNβ as it is used herein refers to all forms of IFNβ including, but not limited to interferon beta-1a and interferon beta-1b. Further, IFNβ as it is used herein includes IFNβ from any source whether endogenously produced, or naturally or synthetically derived, including IFNβ produced by modified microorganisms. In some embodiments, the treatment may stimulate endogenous IFNβ using compositions that include live bacterium or oligosaccharides alone or in various combinations that correct for gut dysbiosis and provide necessary signals to favor IFNβ production. In other embodiments, the treatment also involves exogenous IFNβ. The exogenous IFNβ may be an intramuscular or intravenous or nebulized form.

Compositions used for the prevention or treatment of such disorders comprises either the administration of IFNβ to an infant at risk for a specific autoimmune disease including food allergy, and/or the endogenous stimulation of the production of IFNβ, if not endogenously produced, may include IFNβ-1a and/or IFNβ-1b; and may come from natural or synthetic sources, including modified microorganisms.

A commensal microorganism is one expected to be found, or has been found, in the intestinal tract of an individual. The microorganism is in a relationship where it derives food or other benefits from the host. A symbiont is a microorganism that has a mutually beneficial relationship with a host. The presence or absence of these commensal or symbiotic organisms may change with age, health status, or consumption of different food and fiber sources. Commensals and/or symbionts may be used as probiotics. Probiotics or live bacterium are microorganisms administered to a host for the purpose of improving any aspect of the health of the host and they may, in certain cases, significantly alter the host's gut microbiome.

A gut or intestinal microbiome is the total community of microorganisms residing in the gastrointestinal tract of an individual. It can include bacteria, yeast, and viruses. Gut dysbiosis may result at any age and means a loss or absence of certain bacteria and bacterial functions that results in a loss of capability, performance in the gut microbiome that may lead to insufficient gut function leading to symptoms of reduced gut and/or overall health of the host. In infants, the absence of certain Bifidobacterium and the loss of the ability to consume HMO is an example of infant gut dysbiosis.

A microbiome may be measured with Next Generation Sequencing (NGS) technology using a sequencing depth to identify the family level, to the species or subspecies level, or to be able to look at specific gene functions (metagenomics) to establish the relative abundance or certain taxa or genes within the total microbiome. Individual genus or species' absolute abundance can be measured by quantitative Polymerase Chain Reaction (qPCR) by using primers specific to the organism in question and normalizing to micrograms of feces or micrograms of DNA. A gut microbiome may be assessed for its overall abundance of

### Bifidobacteriaceae and/or Enterobacteriaceae

Any mammal may be susceptible to viral infections. A virus is defined by the type of nucleic acid it uses to replicate in a living host cell, in this case mammalian cells. A virus may also be defined by whether or not it leads to an acute or chronic infection and the type of tissue it infects (i.e. skin, respiratory, gastrointestinal). A virus may be composed of DNA (deoxyribonucleic acid) or RNA (ribonucleic acid) surrounded by a protein coat. A viral infection can lead to a spectrum of symptoms from asymptomatic (no overt symptoms) to severe disease (Merck Manual).

For illustrative purposes, examples of viruses are provided but are not limiting. Any methods and compositions may be used for any known or yet to be known virus as a treatment modality or option where resolving gut dysbiosis and/or producing IFNβ may be of benefit. DNA viruses include herpes viruses. Herpes viruses include for example cold sores, chickenpox and shingles. Chickenpox is an example of a skin virus, rotavirus is an example of a gastrointestinal virus causing gastroenteritis, and respiratory viruses which cause pneumonia can include influenza, corona virus, or respiratory syncytial virus. RNA viruses include retroviruses, such as human immunodeficiency (HIV), and coronaviruses, such as SARS-CoV2 that causes COVID-19. RNA viruses, particularly retroviruses, are prone to mutate, meaning the set of genetic instructions that contain all the information that the virus needs to function, can change as the virus spreads leading to new variants.

### Compositions Comprising a Live Bacterium

In some embodiments, the composition or medicament comprises one or more commensal organism in the form of a live bacterium or probiotic. The commensal organism may be selected from the group comprising the genus of Bifidobacterium or Lactobacillus. Bifidobacterium species may be selected from species such as B. adolescentis, B. animalis, B. animalis subsp. animalis, B. animalis subsp. lactis, B. bifidum, B. breve, B. catenulatum, B. longum, B. longum subsp. infantis, B. longum subsp. longum, B. longum subsp. suis, B. pseudocatanulatum, and B. pseudolongum. Lactobacillus may be selected from species such as L. acidophilus, L. antri, L. brevis, L. casei (or Lacticaseibacillus casel), L. coleohominis, L. crispatus, L. curvatus, L. fermentum, L. gasseri, L. johnsonii, L. mucosae, L. pentosus, L. plantarum (Lactiplantibacillus plantarum), L. Reuteri (Limosilactobacillus reuteri), L. rhamnosus (Lacticaseibacillus rhamnosus), L. sakei, L. salivarius (Ligilactobacillus salivarius), L. paracasei (Lacticaseibacillus paracasei), L. kisonensis., L. paralimentarius, L. perolens, L. apis, L. ghanensis, L. dextrinicus, L. shenzenensis, and L. harbinensis. One skilled in the art will recognize the bacteria is the same even if it has or undergoes a name change in the future.

In some embodiments, one or more Bifidobacterium species may be selected as the live bacterium. The Bifidobacterium may be selected from, but not limited to, the following species: B. adolescentis, B. animalis, B. animalis subsp. animalis, B. animalis subsp. lactis, B. bifidum, B. breve, B. catenulatum, B. longum, B. longum subsp. infantis (B. infantis), B. longum subsp. longum (B. longum), B. longum subsp. suis. B. pseudocatanulatum, and B. pseudolongum. The Bifidobacterium species or subspecies may be selected from the group that are typically associated with infants, such as B. infantis, B. breve and/or B. bifidum. In some embodiments, the infant Bifidobacterium species may be one that has transport mechanisms to internalize intact HMO (e.g., B. infantis) or ones that produce extracellular catabolic enzymes, (e.g., B. bifidum) or ones with both of these capabilities (e.g., B. breve).

In preferred embodiments of this invention the Bifidobacterium present may be an activated Bifidobacterium culture and may be dried prior to administration. (WO 2016/065324 published Apr. 28, 2016 and WO 2019/143871 published Jul. 25, 2019) (incorporated herein by reference). In preferred embodiments of this invention the Bifidobacterium present, activated or otherwise, is B. infantis EVC001 deposited under ATCC Accession No. PTA-125180. In preferred embodiments, the B. infantis is an H5 positive strain or a strain with a fully functional H5 gene cluster in its genome. The H5 positive strain refers to the ability of the strain to transport and use certain oligosaccharide structure (WO 2019/232284). In preferred embodiments, the B. infantis or other live bacterium have specific enzymes, such as or equivalent in function to, EndoBI-1 and/or EndoBI-2 (N-linked endoglycosidases found in B. infantis) to cleave glycans from whey protein in vivo.

In some embodiments, the composition further comprises one or more Lactobacillus. The Lactobacillus may be selected from the group consisting of food-associated Lactobacillus: L. acidophilus, L. brevis, L. casei, L. crispatus, L. curvatus, L. fermentum, L. pentosus, L. plantarum, and L. sakei or one or more Lactobacillus may be selected from the group consisting of host-associated Lactobacillus: L. antri, L. coleohominis, L. gasseri, L. johnsonii, L. mucosae, L. reuteri, L. rhamnosus, and L. salivarius. In some embodiments, the Lactobacillus is L. rhamnosus or the LGG strain or an L. reuteri.

In any of the foregoing embodiments, the composition may comprise live bacterium in an amount of 0.1 million-500 billion Colony Forming Units (CFU) per gram, per serving, or per unit dose delivered as part of a composition. The composition may be in an amount of 0.001-100 billion Colony Forming Units CFU, 0.1 million to 100 million, 1 million to 5 billion, or 5-20 billion CFU per gram, per serving, or per unit dose delivered as part of a composition. The live bacteria may be in an amount of 0.001, 0.01, 0.1, 1, 5, 15, 20, 25, 30, 40, 45, or 50 billion CFU per gram, per serving or per unit dose delivered to the patient as part of a composition. The live bacterium may be in an amount of 5-20 billion CFU per gram, per serving size or per unit dose of the composition or 5-20 billion CFU per gram of composition or 0.1 million to 100 million CFU per gram of composition. In some embodiments, the live bacterium is a Bifidobacterium in an amount of 0.1 million-500 billion Colony Forming Units (CFU) per gram, per serving or per unit dose of a composition at the time of use. The composition may be in an amount of 0.001-100 billion Colony Forming Units CFU, million to 100 million, 1 million to 5 billion, or 5-20 billion CFU per gram, per serving or per unit dose of composition at the time of use. The Bifidobacterium may be in an amount of 0.01, 0.1, 1, 5, 15, 20, 25, 30, 35, 40, 45, or 50 billion CFU per serving, per gram or per unit dose of composition at the time of use. The Bifidobacterium may be in an amount of 5-20 billion CFU per gram of composition or 5-20 billion CFU per gram of composition or 0.1 million to 100 million CFU per gram, per serving or per unit dose of composition at the time of use.

Alternatively, the organism may be a recombinant organism, or an organism selected from the above list that possesses key functions demonstrated by the study of B. infantis that promote IFNβ expression in the human host.

### Compositions Comprising a Mammalian Milk Oligosaccharide

“Mammalian milk oligosaccharides” (MMO) or milk glycans is defined here as any oligosaccharide that exists naturally in any mammalian milk whether it is its free form or bound to a protein or lipid or released from said protein or lipid. Dietary fiber is carbohydrate (e.g., oligosaccharides) that are not catabolized by mammalian enzymes. MMO may make up all or a portion of the dietary fiber in a mammals diet. MMO includes synthetic structures as well as those extracted, enriched, or purified from sources other than mammalian milk so long as the compound mimics that found in mammalian milk in structure and/or function. That is, while MMOs may be sourced from mammalian milk, they need not be for the purposes of this invention. Sources of MMO may include colostrum products from various animals including, but not limited to cows, sheep, goats and other commercial sources of colostrum or milk. It may include derivatives of whey permeate that contain MMO enriched or partially purified, human milk products that are modified through processes such as skimming, protein separation, pasteurization, retort sterilization may also be a source of MMO. MMO includes human milk oligosaccharides (HMO). In preferred embodiments, the MMO is an HMO. MMO may also be referred to as a prebiotic, a glycan, an oligosaccharide and may be selected for its ability to selectively grow certain bacteria in this application, but not others. A suitable oligosaccharide or mixture of oligosaccharides are oligosaccharides that are consumed by the live bacteria selected as part of a given composition. A synthetically produced HMO may be used alone or in combination with mammalian milk sources comprising MMO within the source.

In various embodiments, a prebiotic oligosaccharide or MMO may be administered, and such oligosaccharide can include one or more of lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3 SLN), 3′-fucosyllactose (3FL), 3′-sialyl-3-fucosyllactose(3 S3FL), 3′-sialyllactose (3 SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof.

In preferred embodiments, the MMO is selected from human milk ologisaccharies (HMO), and the HMO may comprise or consist of Lacto-N-tetraose (LNT) and/or Lacto-N-neotetraose (LNnT). In many embodiments, the LNT and LNnT are in the range of 10:1, 4:3, 5:1, 2:1, 1:1 1:2, 3:4 or 1:10 ratio. In some embodiments, the HMO contains at least one sialic acid containing oligosaccharide structure that is not sialyl lactose, at least one fucose containing oligosaccharide that is not fucosyl lactose and either LNT or LNnT. In yet other embodiments, the MMO has at least 2 MMO selected from 2′FL 3′FL, 3′SL, 6′SL LNT, LNnT, DFL and LNFP.

In yet other embodiments, GOS, FOS, PDX or resistant starch is included in the compositions. The ratio of GOS:MMO may be 1:20 to 20:1, or 1:10 to 10:1 ratio with any one of the MMO described herein. In some embodiments, the MMO comprise LNnT or a related structure and 2FL in 1:5 to 5:1 ratio or LNT or a structure related to LNT and 2′FL in 1:5 to ratio Other MMO include but are not limited to trifucosyllacto-N-hexaose (TFLNH), LnNH, lacto-N-hexaose (LNH), lacto-N-fucopentaose III (LNFPIII), monofucosylated lacto-N-Hexose III (MFLNHIII), Monofucosylmonosialyllacto-N-hexose (MFMSLNH). In some embodiments, the GOS preferably has a degree of polymerization (DP) of larger than at least 4 (DP4), DP5 or DP6. In some embodiments, the DP4 is at least 30% of the total GOS provided. In others D4 and D5 make up at least 50% of the GOS Composition. In some embodiments, the GOS has less than 10% DP3 (WO 2010/105207, published Sep. 16, 2010 incorporated herein by reference). In some embodiments, a ratio of GOS/FOS, GOS/inulin, GOS/FOS/inulin, GOS/PDX is used with one or more mammalian milk oligosaccharides. In other embodiments, these are expressly excluded from compositions or from the diet.

In some embodiments, there are at least 2 or at least 3 synthetic oligosaccharides. In some embodiments, the added total dietary oligosaccharides can come from a combination of partially purified oligosaccharide from human milk products, human donor milk, bovine, caprine, or human or bovine glycoproteins, and synthetic single source. Human milk products modified through processes such as skimming, protein separation, pasteurization, retort sterilization may also be a source of MMO for use in any composition described herein.

### Compositions and Medicaments for Stimulating Endogenous IFNβ

Any composition described herein for elevating IFNβ levels in the gut of a mammal may take the form of a powder, an oil suspension, gel, or a liquid. It may be a loose powder (in a sachet or package), in a tablet, in a dissolving tablet, or in a capsule or integrated in a nutritional source depending on the application.

A composition for elevating IFNβ levels in the gut of a mammal provides a live bacterium and a source of MMO. The composition may be used to supplement an existing nutrient source to achieve a certain amount of live bacteria and a certain amount of MMO to reach a minimum total daily intake of the composition, or a certain serving size. One or more servings per day may be required to elevate the IFNβ levels. Any composition may be used to supplement human milk, donor milk, human milk fortifier (human or bovine origin), any mammalian milk, infant formula, follow-on formula, a prepared food, or a meal replacer for an adult. A composition may be part of a complete nutrition source, such as infant formula, follow-on formula, meal replacer, formula for enteral feeding, a prepared food or beverage. The food may be a medical food suitable only for meeting the nutritional needs of the patient population being treated.

The composition may be used as a pharmaceutical agent, in a pharmaceutical preparation, such as an oral treatment, or an enema treatment.

In some embodiments, a composition is used as an adjuvant or complementary treatment to drug therapy for a particular disease or condition. In some embodiments, the adjuvant or complementary treatment increases the lifetime use of the drug or delays mammal becoming resistant to a drug commonly used for the treatment of such a condition.

In some embodiments, the live bacteria may be administered prior, during or after another aspect of treatment. The live bacteria may be administered sequentially or contemporaneously with an MMO. The MMO may be selected specifically as a prebiotic that is consumed by the live bacteria being administered. In some embodiments, administration of live bacteria is sufficient to increase endogenous IFNβ. In some embodiments, the live bacteria adds missing or deficient metabolic functions to the gut microbiome of the subject in need of elevating the level of interferon beta in their gut.

In some embodiments, the mammal or subject may have gut dysbiosis and administration of live bacteria is sufficient to increase endogenous IFNβ. In some embodiments, dietary sources of MMO are sufficient to provide a food source to be consumed by the live bacteria. In some embodiments, the live bacteria or commensal organisms are provided to an individual who has a diet rich in MMO; this may include human milk, human milk products, alone or in combination with formula or both. The MMO may be oligosaccharides selective for B. infantis. In some embodiments, a composition comprising at least one HMO and at least B. infantis is used. In some embodiments, a subject will consume at least 4 gram/liter (g/L), at least 8 g/L, at least 12 g/L, at least 15 g/L, or at least 20 g/L of one or more oligosaccharides chosen to promote the growth of one or more commensal organisms or live bacteria and/or reach a daily intake of at least 1 gram (g), at least 4 g, at least 8 g, at least 12 g, at least 15 g, at least 20 g, at least 25 g, at least 30 g, at least 35 g, at least 40 g, at least 45 g, at least 50 g, or more than 50 g.

In some embodiments suitable for infants, infant formula is carefully formulated to provide only oligosaccharides selective for or favoring the growth of B. infantis over other live bacteria including other Bifidobacterium. In other words, the methods and compositions of this invention are suitable for use in or for use with formula's that do not contain galacto-oligosaccharides (GOS), Fructo-oligosaccharides (FOS), short or long chain inulin, maltodextrin, or polydextrose (PDX). B. infantis may stabilized and added to that formula or provided in a separate package to add to formula at the time of consumption from either a powdered form, or from a form suspended in MCT oil.

Any live bacteria and MMO composition may comprise a particular protein source such as whey or soy. Proteins may be partially or extensively hydrolyzed, or may be in the form of amino acids, such as, but not limited to taurine, leucine, and tryptophan. Soy protein may be intact or in peptide form. In some embodiments, the formulation comprises a glycosylated whey protein. The glycosylated whey protein may be from whey concentrate or whey isolate produced from colostrum or mature milk. The whey protein may be a base for a specialized food. In some cases, the base has an amount of whey greater than that of casein. In other embodiments, the whey is partially hydrolyzed. The whey may be human or bovine. In some embodiments, the diet comprises at least a source of tryptophan.

Any composition may also comprise soy lecithin. In other embodiments, compositions further comprise indole lactate or other tryptophan derivatives or metabolites.

In some embodiments, the compositions may further comprise minerals such as, but not limited to calcium phosphate, and/or selenium.

In yet other embodiments, compositions may be mixed with ingredients comprising oils such as but not limited to MCT oil, palm olein, soy oil, coconut oil, high oleic sunflower oils, and oils rich in docosahexaenoic acid (DHA) and/or arachidonic acid (ARA).

In some embodiments, the compositions may be mixed with ingredients comprising vitamins such as, but not limited to, vitamin A palmitate or provitamin A, vitamin D3, vitamin E acetate, and/or vitamin K.

In some embodiments, compositions may further comprise lactose, sialic acid, fucose, glucose and/or galactose.

In some embodiments, a composition further comprises a milk fat globule membrane (MFGM) complex. Compositions that include MFGM may be chosen from those in PCT/US2020/043756 incorporated by reference.

In some embodiments, the formulations comprise B. infantis and/or MMO and one or more allergenic proteins, such as those from milk, egg, soy, wheat, shellfish, tree nut, peanut, fish or sesame, that are known to cause allergic reactions in susceptible individuals. In some embodiments, the amount of allergen is less than 1 mg, less than 5 mg, less than 10 mg, less than 20 mg, less than 30 mg, less than 40 mg, less than 50 mg, less than 60 mg, less than 100 mg or more than 100 mg. In some embodiments, the allergen is added in increasing amounts over time or in stages wherein the diet is specified to the amount of allergen provide as a total daily intake.

In some compositions, the live bacteria are dried and then suspended in an oil, such as a MCT oil or other oils described herein. The oil may further comprise DHA and/or ARA, and/or fat-soluble vitamins, such as a vitamin A, provitamin A and/or Vitamin D.

In some compositions, an appropriate concentration of HMO is delivered in a sterile liquid such as water. The HMO water is pre-mixed to have a concentration of 1 g/L, 2 g/L, 3g/L, 4 g/L, 8 g/L, 10 g/L, 12 g/L, 15 g/L , 20 g/L, 30 g/L or 40 g/L. An HMO water may be fed or administered throughout the day to reach a desired total dietary intake appropriate for the age and application used to prevent or treat a condition in a mammal. In some cases, the HMO may be a powder or a paste that is added to any food to reach a daily intake between 1-50 grams of one or more of the MMO described herein.

In compositions where the HMO is in a sterile water, the volume fed modifies the amount of HMO delivered, based on age and/or need. In some instances, the HMO water is used to maintain MMO concentration in diet of at least 4 g/L, at least 8 g/L, at least 12 g/L at least 15 g/L or at least 20 g/L. In some instances, the MMO formulation maintains dietary intake of at least 5 g, at least 10 g, at least 15 g, at least 20 g, at least 30 g, or at least 50 g per day. In some embodiments, the MMO may be in the form of an HMO water, paste, or a powder, while the live bacteria may be in an oil suspension and these two components may be combined prior to or contemporaneously with administration.

Compositions disclosed herein may comprise a source of extra-intestinal or exogenous IFNβ alone, or in combinations with a Bifidobacterium species and/or one or more oligosaccharide. The exogenous IFNβ may be in a form for oral, rectal, intravenous, intramuscular or in a nebulizer form.

### Use of Compositions

Any of the compositions of this invention may be applied to a mammal in a form suitable to that mammal's age, stage of life, diet, and/or clinical or therapeutic need. A mammal may be a human, a non-human primate, a food production animal, such as, but not limited to, a cow, pig, goat, sheep, buffalo, horse, or a performance or domesticated animal, such as, but not limited, to a horse, dog, or cat.

In some embodiments, the method comprises delivering a unit dose, a medicament, a food, a medical food, a dietary supplement, or a pharmaceutical agent. The composition may be delivered as part of a dietary regimen specific to the needs of the mammal during treatment. The food may be an infant food, or a food for resolving inflammation or preventing acute viral infection, food for supporting or managing chronic viral infections, food for prevention or treatment of food allergy, food to prevent or support a mammal with an autoimmune or other allergic disease.

The live bacterial composition in oil may be mixed with any milk, infant formula, water, or consumed directly. In a different method, the live bacteria and MMO are combined as powder. The method may involve calculating a CFU unit dose or serving per gram of MMO delivered to determine an effective dose to elevate IFNβ. In some embodiments, the MMO is metered out throughout the day to deliver a constant source of MMO to the gut.

In some embodiments, the method involves administering the live bacteria alone or in combination with MMO as a food, an infant food, a medical food, a dietary supplement, or a pharmaceutical agent. In other embodiments, the composition further comprises other food or pharmaceutical agents that may have other components to elevate the level of interferon beta in the gut of a mammal. The live bacteria and/or an oligosaccharide as selected from the bacterial species or the MMO structures described herein may be administered contemporaneously or sequentially as needed to reach an effective amount of IFNβ.

In some embodiments, the mammal has or is at risk of one or more of the following conditions: an acute viral infection, a chronic viral infection and autoimmune or allergic disease. A method of treating these conditions may further comprise additional food, medical food, dietary supplement or pharmaceutical agents and may be achieved with any composition described herein.

In some embodiments, the method for the treatment or prevention of gut dysbiosis associated with autoimmune and allergic disease comprises stimulating a therapeutically effective dose of IFNβ to a mammal suffering from, or at risk of suffering from such a disease.

In some embodiments, gut dysbiosis may be an underlying risk factor for severity of viral infections, or may be associated with autoimmune and allergic disease. Any allergy or autoimmune disorder that can be reasonably traced to gut dysbiosis may be treated using a method and composition described herein. In other embodiments, the composition may be used as a mechanism to increase IFNβ regardless of presence of absence of underlying gut dysbiosis. Stimulation of IFNβ may induce immune tolerance, resolve inflammation, or reduce the severity of co-morbidities associated with acute inflammatory insults.

The composition may be administered orally or rectally to a hospitalized patient, optionally the administration of oral, nebulized, intramuscular or intravenous recombinant IFNβ may be included as part of a treatment protocol. In other embodiments, the oral regimen is started subsequent to an acute treatment protocol that might involve steroids and/or recombinant IFNβ. In some embodiments, the composition may be used to resolve the sequalae of acute infection and inflammation. In some embodiments, the method reduces co-morbidity and/or mortality associated with infection.

A method to prevent or treat an autoimmune or allergic condition or disease comprises administering a live bacterium and MMO. Autoimmune and allergic conditions may include, but are not limited to, rheumatoid arthritis, psoriasis. multiple sclerosis, celiac disease, inflammatory bowel diseases (Crohn's, ulcerative colitis,) inflammatory bowel syndrome (IBS), Type 1 diabetes mellitus, idiopathic pulmonary fibrosis, atopic dermatitis, asthma, and food allergies.

Treatment serves to protect and enhance barrier properties during acute and chronic illness or serves as a background to introduce allergens to infants at risk of allergy.

In specific embodiments, the method is intended to administer compositions specifically to prevent or treat food allergy. In some embodiments, the method comprises delivering B. infantis and/or MMO to increase IFNβ prior to administration of an allergen. In another embodiment, B. infantis, MMO and one or more allergens, such as milk, egg, soy, wheat, shellfish, tree nut, peanut, fish or sesame, that are administered contemporaneously or sequentially. In some embodiments, the amount of allergen is less than 1 mg, less than 5 mg, less than 10 mg, less than 20 mg, less than 30 mg, less than 40mg, less than 50 mg, less than 60 mg, less than 100 mg or more than 100 mg. In some embodiments, the allergen is added in increasing amounts over time.

In some embodiments, elevating the level of IFNβ in the gut of the mammal for the treatment or prevention of gut dysbiosis in mammals with acute or chronic viral infections comprises stimulating a therapeutically effective amount of IFNβ. The therapeutically effective amount may be through stimulation of endogenous IFNβ and treatment may further comprise exogenous IFNβ. Exogenous IFNβ may be delivered by any means, orally, rectally, intramuscularly, intravenously or through a nebulizer.

Chronic viral infections can include, but are not limited to, HIV, herpes, hepatitis. Acute viral infections include, but not limited to, respiratory syncytial virus (RSV), influenza type viruses, and the Coranaviridae family of viruses that includes COVID 19 and its variants. One skilled in the art will recognize that these methods and compositions can be used as a treatment option for any viral disease whether or not the virus or family of viruses is explicitly named here. The live bacteria and MMO may be an anti-viral medicament used in mammals exposed to said virus. It may be used in patients exposed but not yet symptomatic to elevate IFNβ. In some embodiments, the method may be used to reduce viral carriage in a mammal suffering from a viral infection, said method provides live bacteria and MMO that are consumed by that live bacteria, to the mammal in need in the same composition.

In any of the forgoing embodiments, a treatment for the prevention, reduction of risk or symptoms, maintaining a subject in remission or prolonging the longevity of effective treatment for a target diseases and conditions may vary due to specific considerations, such as age and diet, and any of the compositions described herein may be applied to meet one of these needs for a mammal. For example, an infant, a preterm infant, a toddler, a child, an adult, an older adult, or a geriatric mammal may require different compositions, doses, duration to achieve an effective therapeutic endpoint.

A method to treat a human mammal may select the live bacteria and MMO combination based on the age of the human. The age may be selected from a preterm infant, a term infant, a toddler, a child, a young adult, an adult, an older adult or a geriatric adult. In general, an infant is defined as 1 year or less, a toddler is 1-3 years of age, a child is 3-12 years of age. An infant is selected from an age group comprising infant 0-3 months, infant 3-6 months, infant 6-12 months, a toddler is 1-3 years, a child is 3-5, 5-10, 1-16 years, a young adult is 16-18 years, an adult is 18+, 20+, 30 +, 40+, 50+, an older adult is 60+, 70 +or a geriatric adult is 80+90+ years.

Any of the compositions may be tailored or targeted to specific infant age groups, such as a preterm infant who may be born with a gestational age of less than 33 weeks, the preterm babies may be an extremely low weight (ELBW), very low birth weight (VLBW), or low birth weight (LBW), a term infant (0-3 months), an infant 3-6 months, an infant (6-12 months), a weaning infant (4-12 months), a weaned toddler (12 months to 2 years). Treatment may be modified if the infant is weaned onto complementary foods that introduce potential allergenic proteins, if the infant has an exclusive human milk diet, an exclusive infant formula, or a mixed diet of human and infant formula. A specific formulation may exist for infants 0-3 months, 3-6 months, 6-12 months or a composition suitable for 0-24 months, but may change if a child was more than 3 years of age.

Administration of such methods and compositions may improve the growth rate of the human infant measured by kilograms/day, Z scores, such as weight for age (WAZ), length for age (LAZ) or (weight for length) WLZ.

In some embodiments, the method provides a composition that protects the gut from opportunistic pathogen invasion (i.e., to provide colonization resistance). Any composition may reduce the load of potential pathogens. The composition may be used to lower the fecal pH to assist with elevating IFNβ.

Infants may require different compositions during the time where their adaptive immune system is developing. In some embodiments, the infant whose adaptive immune system is developing may be a human preterm infant who needs to reach key developmental milestones for growth, reduced risk of viral infection, such as RSV, or necrotizing enterocolitis, or late onset sepsis, and therefore needs B. infantis and specific HMO to reduce or resolve inflammation. The infant whose adaptive immune system is developing may be a term infant—age 0-3, 0-6, 6-12, 21-24 or 0-24 months — who needs at dietary intake of MMO of at least 1 g/L, at least 2 g/l, at least 4 g/L, at least 6 g/L, at least 8 g/L, at least 10 g/L, at least 12 g/L, at least 15 g/L or at least 20 g/L.

In some embodiments, compositions are used to lower the pH and increase colonization resistance, decrease the level of Enterobacteriaceae in the gut of a non-infant at a time when the subject is in need of mucosal healing (WO 2017/156550 incorporated by reference) as a step in treatment to increase IFNβ levels in a hospitalized subject or one that has suffered from an acute viral infection. In some embodiments, compositions are used to reduce gut dysbiosis causing intestinal distress or mucosal damage. Gut dysbiosis may be reduced by reducing carriage of antibiotic resistant genes and/or levels of endotoxin and/or chronic gut inflammation and/or increased IFNβ at a time when the subject is in need of mucosal healing. In some embodiments, the composition is used to protect the intestinal mucosa during treatment to promote wound healing and prevent leaky gut.

In some embodiments the method of use requires that the diet of the patient comprises a source of protein that includes glycosylated whey protein and a good source of tryptophan. The diet may also include sources fortified with Vitamin A and/or Vitamin D. The composition and/or diet may contain sources of tryptophan, serotonin and/or melatonin.

In some embodiments, compositions comprising species of Bifidobacterium (most notably B. infantis) are used to modulate the levels of IFNβ in the infant gut, thereby improving gut health and reducing the likelihood of developing autoimmune disorders or allergies.

In some embodiments, an individual with a chronic inflammatory or autoimmune disorder will be administered foods that provide a rich source of tryptophan and/or indole lactate, glycosylated whey protein (intact or partially hydrolyzed), at least one commensal organism, and one or more sources of MMO to make up increasing amounts of the total dietary fiber. In some instances, the other dietary fiber may need to be reduced.

In some applications, the diet of the mammal is modified to reduce or eliminate other sources of dietary fiber in the diet while mammal undergoes treatment, while increasing the MMO content of the diet to 1-50 gram per day.

In some embodiments, recombinant IFNβ may be used prior to, or contemporaneously, with B. infantis and/or MMO to promote intestinal barrier function, improve feeding tolerance. In some embodiments, the subject in need of such a composition is in the neonatal intensive care (NICU), pediatric intensive care (PICU) or intensive care (ICU).

Any of the compositions described herein are provided daily for at least 1 day, at least 3, at least 7, at least 14, at least 28 days, at least 3 months, at least 6 months or at least 12 months to any subject in need of.

In infants, methods to prevent autoimmune or allergic disease administration of live bacteria start with maternal administration, given to infant at birth, within first week of life, in the first 100 days of life, the first 6 months of life or in the first year of life wherein the compositions are provided daily for at least 1 day, at least 3 days, at least 7 days, at least 14 days, at least 28 days, at least 2 months, at least 3 months, at least 6 months or at least 12 months.

The present invention contemplates methods of treating patients described herein, the methods requiring administering compositions described in WO 2018/006080, and WO 2019/232513, the disclosures of which are incorporated by reference herein in their entireties.

### Measuring IFNβ—Diagnosis and Monitoring

IFNβ levels either in stool or blood may be monitored to evaluate therapeutic efficacy. [Henrick, B. M. et al (2019). Colonization by B. infantis EVC001 modulates enteric inflammationin exclusively breastfed infants. Pediatr Res 86, 749-757]. The IFNβ may be measured enterically or systematically, from stool or blood samples, and the measurement is used to diagnose need and/or monitor effectiveness of treatment. A therapeutically effective dose is any dosage of IFNβ which has the effect of preventing or reducing acute symptoms caused by a gut dysbiosis associated autoimmune or allergic disease or a viral infection. The absence of IFNβ below a threshold may indicate need for treatment, or modified treatment. Conversely, the presence of IFNβ above a threshold indicates a composition and method that successfully favored to production of IFNβ and may be deemed therapeutically effective, as this level may also coincide with additional clinical signs of resolution of inflammation. In some embodiments, measurement is used to modify treatment in response to measurement.

The use of any composition may enhance the growth and colonization of live bacteria as a step in elevating IFNβ. In a preferred embodiment, the use of at least one Bifidobacterium species and more preferably B. infantis, such that the relative abundance of the Bifidobacterium family (Bifidobacteriaceae) increases to at least 25%, at least 30%, at least 40%, at least 50%, at least 65%, at least 75%, at least 85%, or at least 90% of the total microbiome; whereas in other embodiments, the Enterobacteriacaeae decreases to less than 30%, less than 20%, less than 15%, less than 10%, less than 5%, or less than 1% of the total microbiome.

At any age, MMO may be varied to increase absolute and/or relative abundance of live Bifidobacterium species detected in stool, to increase fecal IFNβ. The MMO levels may be modified by measuring MMO in stool. The sufficiency of MMO to meet needs of live bacteria or commensal organisms may be determined by measuring and monitoring levels of MMO in the stool. In a preferred mode, the bacteria capable of consuming the MMO are present in amounts sufficient to reduce the residual MMO passing into stool to low levels.

Monitoring and maintaining IFNβ levels, as well as other cytokines, can be used to tailor and personalize treatment to achieve the necessary clinical improvements. For example, subjects with conditions such as, but not limited to rheumatoid arthritis and psoriasis need to monitor for increases in IFNβ and decreases in IL-7, IL-21 and IL-22. Subjects with food allergy and other allergic diseases need to monitor for increases in IFNβ production and decreases in IL-4, IL-5 and IL-13 as well as antigen specific IgE reactions enterically and/or systemically. For subjects with HIV infection, elevated IFNβ production dampens immunopathogenesis, including enteric and systemic inflammation, indicative of HIV-1 progression of disease towards AIDS.

### EXAMPLES

### Example 1. Stimulation of IFNβ by Daily Feeding of B. infantis EVC001 in Newborn Term Infants

Study design. A total of 120 fecal samples (ClinicalTrials.gov: NCT02457338;

clinicaltrials.gov/ct2/show/NCT02457338) from 40 different subjects were analyzed at three time points—day 6 (Baseline), day 40, and day 60 postnatally. Individual subjects were chosen at random and made up a subset of the original study participants. All aspects of the study were approved by the University of California Davis Institutional Review Board (IRB Number: ID 631099) and all participants provided written informed consent. Briefly, exclusively breastfed term infants were randomly selected to receive 1.8×10¹⁰ colony- forming units (CFU) B. infantis EVC001 daily for 21 days (EVC001) starting at day 7 postnatal or to receive breast milk alone (control) and followed up to postnatal day 60 [Frese, S. A., et al. (2017). Persistence of Supplemented Bifidobacterium longum sub sp. infantis EVC001 in Breastfed Infants. Msphere 2, e00501-17]. All mothers received lactation support throughout the study. The demographic information (e.g., age, sex, and gestational age) was collected from each participant. Here stool samples from randomly selected infants who were fed EVC001 (n=20) and control infants (n=20) on days 6 (Baseline), 40, and 60 postnatal were collected and analyzed for multiple proinflammatory cytokines using multi-plexed immunoassays and levels of fecal calprotectin with an enzyme-linked immunosorbent assay (ELISA).

Bacterial DNA Methods . DNA was extracted from 296 stool swab samples stored in DNA/RNA shield lysis tubes (Zymo Research, Irvine CA) using the ZymoBIOMICS 96 MagBead DNA kit (Zymo Research). Extracted DNA was quantified using QuantIT dsDNA Assay kit, high sensitivity (ThermoFisher Scientific, Waltham, MA) according to the manufacturer's protocol. 3 samples were omitted from downstream analysis due to failure to meet input requirements for library preparation. Libraries were prepared for each sample using the Illumina Nextera DNA Flex library kit (Illumina, San Diego, CA) with unique dual indexes according to manufacturer guidelines. Libraries were pooled and submitted to UC Davis DNA Technologies core for sequencing on the Illumina NovaSeq S4 flow cell. (Illumina, San Diego, CA). Each lane of the S4 flow cell contained 96 libraries.

Absolute quantification of B. infantis by Quantitative Real-Time PCR. Quantification of the total B. infantis was performed by quantitative real-time PCR using Blon_2348 sialidase tgene primers Inf2348F (SEQ ID NO: 1,5′-ATA CAG CAG AAC CTT GGC CT-3′) (SEQ ID NO: 2,5′-GCG ATC ACA TGG ACG AGA AC-3′), and Inf2348_P (SEQ ID NO: 3,5′-/56-FAIM/TTT CAC GGA/ZEN/TCA CCG GAC CAT ACG/31ABkFQ/-3′) [Frese, S. A. et al. (2017). Persistence of Supplemented Bifidobacterium longum subsp. infantis EVC001 in Breastfed Infants. Msphere 2, e00501-17]. Each reaction contained 10 μL of 2× TaqMan Universal Master Mix II with UNG master mix (Applied Biosystems), 0.9 μm of each primer, 0.25 μM probe and 5 μL of template DNA. Thermal cycling was performed on a QuantStudio 3 Real-Time PCR System and consisted of an initial UNG activation step of 2 minute at 50° C. followed by a 10 minute denaturation at succeeded by 40 cycles of 15 s at 95° C. and 1 min at 60° C. Standard curves for absolute quantification were generated using genomic DNA extracted from a pure culture of B. infantis EVCOOL Quality filtering and removal of human sequences. Demultiplexed fastq sequences were quality filtered, including adaptor trimming using Trimmomatic v0.36 with default parameters [Bolger, A. M., Lohse, M., and Usadel, B. (2014). Trimmomatic: a flexible trimmer for Illuminasequence data. Bioinformatics 30, 2114-2120]. Quality-filtered sequences were screened to remove human sequences using GenCoF v1.0 [Czajkowski, M. D., Vance, D. P., Frese, S. A., and Casaburi, G. (2018). GenCoF: a graphical userinterface to rapidly remove human genome contaminants from metagenomic datasets. Bioinformatics 35, 2318-2319] against a non-redundant version of the Genome Reference Consortium Human Build 38, patch release 7 (GRCh38_p7; nebi.nlm.nih.gov). Human sequence-filtered raw reads were deposited in the Sequence Read Archive (SRA; ncbi.nlm.nih.gov/sra) under the reference number, PRJNAXXXX.

Multiplexed Immunoassays. Interleukin (IL)-1β, IL-2, IL-4 IL-5, IL-8, IL-10, IL-13, IL-17A, interferon (IFN) γ, and tumor necrosis factor (TNF) α were quantified from mg of stool diluted 1:10 in Meso Scale Discovery (MSD; Rockville, MD) diluent using the U-PLEX Inflammation Panel 1 (human) Kit according to the manufacturer's instructions as previously published (ref). Standards and samples were measured in duplicate and blank values were subtracted from all readings. The concentration of fecal calprotectin and 1VIPO were quantified using MSD R-PLEX Human Calprotectin Antibody Set. The samples were plated in duplicate and the assay was performed twice. The plates were then read on a Sector Imager 2400 MSD Discovery Workbench analysis software.

Statistical Analysis. All statistical analyses were performed in R v3.6.2. A Kruskal-Wallis one-way analysis of variance coupled with an FDR or Bonferroni correction was used for statistical comparisons between individual genes, cytokines and taxa amongst groups. Statistical analysis to assess total resistome or enterotype composition by group was performed using a Mann-Whitney or Holm—adjusted Dunn's test. Rarefaction curves were computed to estimate the diversity of the identified ARGs across samples. A nonparametric two-sample t-test was used to compare rarefaction curves using Monte Carlo permutations (n=999). Enterotype analysis was performed as previously described (Arumugam, M. 2011). Cytokines and B. infantis-specific qPCR were correlated with the Spearman method with FDR correction. The P-values throughout the example are represented by asterisks (*, P<0.05; **, P<0.01; ***, P<0.001; ****, P<0.0001).

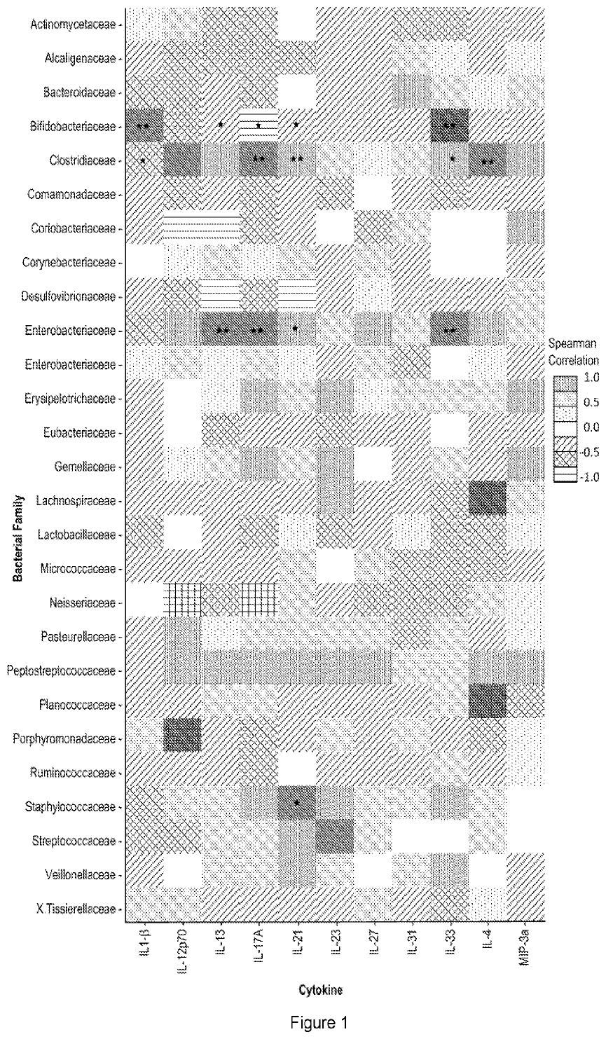

Gut microbiome composition correlates with the production of proinflammatory cytokines and calprotectin (Henrick 2019; Rhoads 2018; Herrera, 2016; Ho, 2019); however, little is known about the relationship with Type I IFN production. We have access to a unique cohort of infants that were all healthy, full term and exclusively breast-fed infants whose only difference was the colonization by Bifidobacterium longum subspecies infantis EVC001. Here, pairwise correlation tests were completed for 40 individual infant stool samples at day 60 of life between the microbial taxonomic composition and specific enteric cytokine concentrations (Spearman correlation with Benjamini-Hochberg false discovery rate correction a<0.02) identified three taxa, including Clostridiaceae, Enterobacteriaceae, and Staphylococceae, significantly correlated with proinflammatory cytokine production. Specifically, Clostridiaceae correlated with IL-21, IL-33, and IL-4, Enterobacteriaceae correlated with IL-13, IL-17A, IL-21, IL-33, and IL-4, while Staphylococceae correlated with higher levels of IL-21. Clostridiaceae and Enterobacteriaceae negatively correlated with IFNβ levels. Conversely, Bifidobacteriaceae abundance was the only taxa that correlated with increased levels of IFNβ and negatively with proinflammatory cytokines IL-13, IL-21, IL-4, MIP1a). Direct comparison between Bifidobacteriaceae abundance and IFNβ show a direct correlation in the heatmap of FIG. 1 (FIG. 1b; P=9.8e-06, rho=0.65), while Enterobacteriaceae and Clostridiaceae abundance negatively correlated with IFNβ concentrations (FIG. 1c and d; P=0.0013, 0.0034 rho=−0.5, −0.46, respectively).

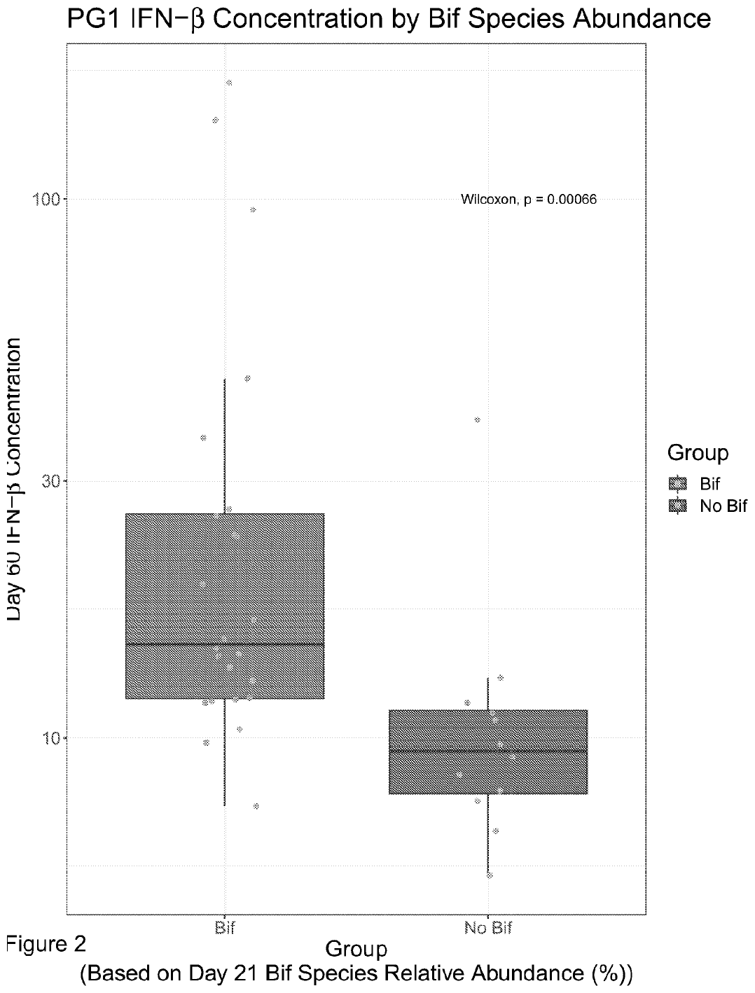

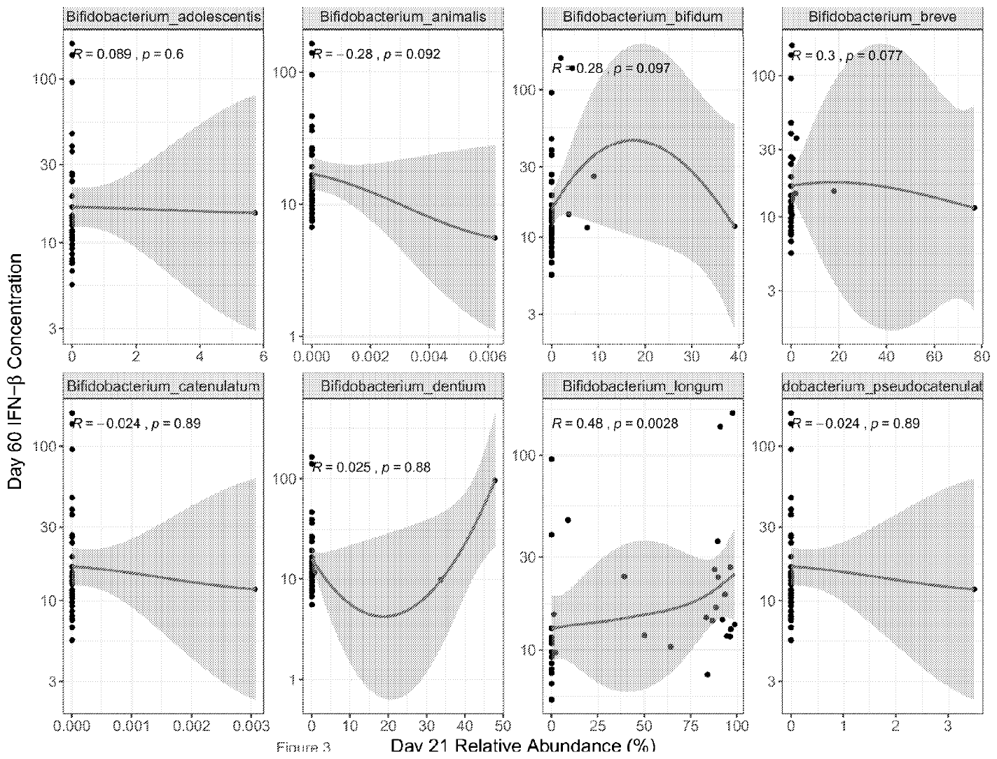

Early microbiome composition can have a dramatic impact on T cell development, vaccine responses, and risk of developing autoimmune and allergic diseases later on in life (Huda, Vatanen, Arrieta); therefore, we next evaluated whether IFNβ concentration at day 60 correlated with early colonization of Bifidobacteriaceae at day 21 postnatal. Fecal samples containing Bifidobacterium at day 21 had significantly increased levels of IFNβ compared to samples that did not contain Bifidobacterium by day 60 postnatal (FIG. 2; P=0.00066). At a species level, B. longum abundance at day 21 correlated with IFNβ levels at day 60 postnatal (FIG. 3; P=0.0028, rho=0.48). Importantly, no other Bifidobacterium species correlated with increased IFNβ levels indicating that B. longum abundance is the main species driving increases in IFNβ production.

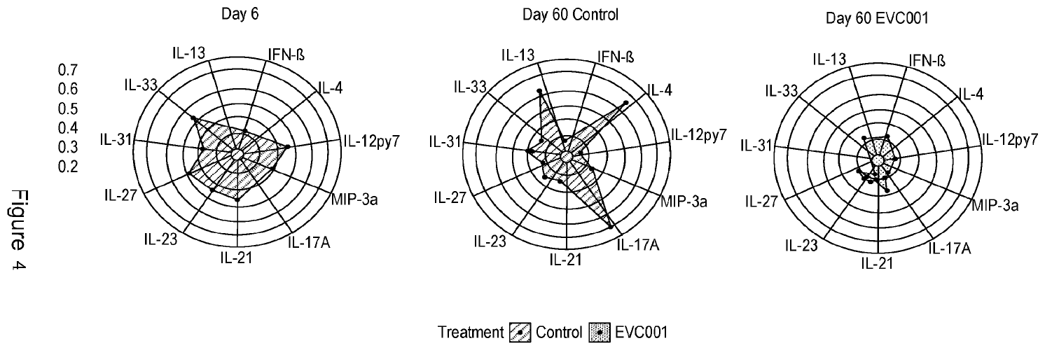

We next sought to determine whether colonization with B. infantis EVC001 could modulate intestinal cytokine production. Baseline represents all forty infants the day before feeding B. infantis commenced. By day 60 postnatal, we observed a significant modulation of the fecal cytokine profiles in the B. infantis-fed infants compared to the infants in the control group. Specifically, fecal concentrations of IL-4, IL-13, IL21, IL-31, and IL-33 were significantly lower in EVC001 infants (FIG. 4, table 1; P=0.038, 0.0065, 0.03, and 0.0003, respectively), while IFNβ levels were significantly increased (FIG. 3, table 1; P=0.047) compared to the controls depicted in radar plot of FIG. 4.

TABLE 1

| | Day 6 (Baseline) | | | Day 60 | | | |
|---|---|---|---|---|---|---|---|
| | Median (SD) [pg/mg] | | | Median (SD) [pg/mg] | | | |
| | | | P | | | P | Fold |
| Cytokine | Control | EVC001 | value | Control | EVC001 | value | Change |
| IFN-β | 14.89 | 13.76 | 0.73 | 11.61 | 17.03 | 0.047 | −1.5 |
| | (91.73) | (18.24) | | (22.41) | (42.63) | | |
| IL-12p70 | 0.29 | 0.22 | 0.27 | 0.14 | 0.12 | 0.41 | 1.2 |
| | (0.21) | (0.54) | | (0.23) | (32.48) | | |
| IL-13 | 10.72 | 29.14 | 0.49 | 35.62 | 25.06 | 0.038 | 1.4 |
| | (8.24) | (25.42) | | (32.89) | (10.91) | | |
| IL-17A | 8.51 | 18.45 | 0.25 | 24.67 | 9.21 | 0.14 | 2.7 |
| | (13.25) | (26.88) | | (26.94) | (9.97) | | |
| IL-21 | 10.07 | 8.98 | 0.17 | 6.15 | 4.5 | 0.0065 | 1.4 |
| | (4.14) | (4.63) | | (10.6) | (0.97) | | |
| IL-23 | 3.33 | 2.59 | 0.53 | 2.16 | 1.58 | 0.14 | 1.4 |
| | (3.35) | (3.7) | | (4.27) | (142.5) | | |
| IL-27 | 41.51 | 32.99 | 0.26 | 20.75 | 18.21 | 0.29 | 1.1 |
| | (21.31) | (23.67) | | (24.6) | (18.14) | | |
| IL-31 | 13.93 | 16.04 | 0.5 | 14.44 | 10.58 | 0.03 | 1.4 |
| | (10.91) | (10.48) | | (3.71) | (3.36) | | |
| IL-33 | 0.8 | 0.67 | 0.31 | 0.42 | 0.26 | 0.0003 | 1.6 |
| | (0.28) | (0.29) | | (0.25) | (0.07) | | |
| IL-4 | NA | 0.69 | NA | 1.49 | 0.69 | 0.08 | 2.2 |
| | (NA) | (0.19) | | (4.46) | (0.66) | | |
| MIP-3α | 0.97 | 0.92 | 0.49 | 0.87 | 0.58 | 0.03 | 1.5 |
| | (0.56) | (0.48) | | (0.91) | (0.23) | | |

### Example 2. Prevention and/or Treatment of Severe Acute Malnutrition

Malnutrition is an ever-present issue worldwide. It is estimated that over 18 million children under the age of 5 are affected by the most extreme form of undernutrition, severe acute malnutrition (SAM). Children with SAM are twelve times more likely to die than well-nourished children. Infectious morbidity is common among survivors. Causes of malnutrition are typically understood to be related to chronic poverty, lack of access to nutritious foods, lack of appropriate breastfeeding, repeated infections and poor hygiene. Although improvements can be made by providing malnourished children with adequate nutrition, there is still a population refractory to current therapeutic interventions. Research indicates that gut microbes are related to undernutrition and that children with SAM have gut dysbiosis that mediates some of the pathology of their condition. The standard of care in these children should be reinforced by an intervention that corrects the gut dysbiosis, barrier function and B and T cell function, improves weight gain during nutritional rehabilitation, and reduces infectious morbidity.

In a Single-blind RCT, stratified randomization study, the T cell response following intervention to modulate the infant gut through administration of B. infantis, LNnT. The target population was first stratified into 2 groups. Group 1 consisted of SAM infants between 2 and <6 months old with severe acute malnutrition randomized to three treatment arms upon completion of stabilization phase of treatment of SAM; SAM was defined as weight-for-length <−3 Z. Group 2 consisted of non-malnourished infants (WLZ≥−1)<6 months old who are hospitalized for treatment with antibiotics for infection. Infants in this group received at least 50% of their nutritional intake from breast milk in order to be eligible for enrollment. Exclusion criteria (for both groups): Septic shock or very severe pneumonia requiring assisted ventilation or acute kidney injury on admission, congenital defects interfering with feeding such as cleft palate, chromosomal anomalies, jaundice, tuberculosis, presence of bilateral pedal edema, maternal antibiotic usage for breastfeeding infants (current antibiotic use). Group 1 (SAM) additional exclusion criteria: Infants receiving ≥75% of nutrition from breast milk. Group 2 (non-malnourished) additional exclusion criteria: Infants receiving <50% of nutrition from breast milk.

The microbiome response to probiotic supplementation (with and without prebiotics) in patient population Group 1 was monitored. Additionally, non-malnourished infants who are hospitalized for infectious conditions face challenges related to dysbiosis caused by antibiotic usage. In Group 2, the ability of activated B. infantis EVC001 to rescue the microbiome of primarily breastfed non-malnourished infants was evaluated.

Stool samples were collected for evaluation of B. infantis colonization and markers of mucosal epithelial monolayer integrity and inflammation. Blood samples were collected at baseline and at 28 days for evaluation of peripheral blood mononuclear cell characterization (including regulatory T cells, B cell and plasma cells) and vaccine response. Babies were evaluated for symptoms or severe acute malnutrition or enteric infections, including the development of sepsis.

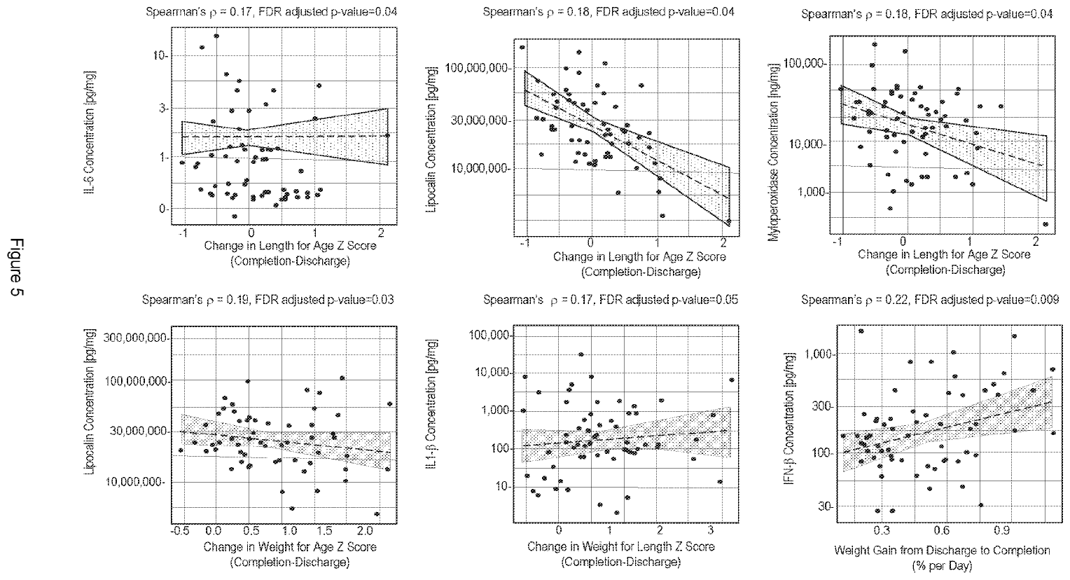

FIG. 5 illustrates that IFNβ levels significantly correlated with weight gain in these infants who are susceptible to infection. Reduced inflammation as measured by myeloperoxidase was associated with improved growth in these infants.

### Example 3. Prevention of Food Allergy in Infants with Family History of Allergy

An infant who has a parent or sibling with severe allergy is identified at birth. The infant is fed 8 billion CFU per day of B. infantis EVC001 starting by day 7 of life. If bovine milk protein allergy is a concern, the infant will be provided human milk, a non-dairy source of tryptophan until suitable B. infantis colonization and/or IFNβ levels are achieved. At such time, the infant, is fed increasing amounts of alpha-S1-casein and/or beta lactoglobulin and monitored for allergic reactions, enteric or systematic IL-4, IL-5 and IL-13, calprotectin, and/or antigen specific and non-specific immunoglobulin E (IgE), antibody titers.

The infant whether fed human milk or infant formula is monitored to maintain at a diet containing at least 15 g/L of HMO. If, the infant is exclusively breast-fed, the infant receives only B. infantis daily unless stool analysis determines insufficient HMO present in stool. At such time, an HMO supplement may be added. If the infant is exclusively formula fed, the infant formula is made up with HMO water to achieve sufficient HMO to maintain B. infantis and IFNβ. If the infant is a mixed feeder, infant formula bottles are made up with FLMO water. Fecal sample analysis: metagenomics, 16S, qPCA, cytokines and calprotectin, antibody titers.

Once the IFN-B levels are established, the addition of potential allergic proteins may be initiated sequentially and treatment stops when tolerance to the protein is achieved.

### Example 4. Treatment of Food Allergy in a Child or Adult with an Established Allergy

An adult or child suffering from food allergy will be measured for gut dysbiosis through fecal analysis of cytokines including but not limited to IFNP, IL-4, IL-5 and IL-13, along with calprotectin, and/or Bifidobacteriaceae, and/or Enterobacteriaceae to develop a treatment plan. The adult will undergo an elimination diet for 2 weeks to remove the allergen and reduce the non-MMO fiber in their system. They will then receive 8 billion CFU each of B. infantis, B. longum and B. breve daily for 14 days and will also consume 20 g of synthetic HMO mixture of LNT, LnNT, 2FL and 6′ SL with 5 g GOS daily during the time of treatment prior to re-introduction of the allergen. Treatment will continue for as long as the food allergy symptoms exist and will be stopped when tolerance is achieved as measured by lack of clinical signs, and/or measurement of IFNβ. Fecal samples may be monitored for cytokines and calprotectin, antibody titers. Blood sample analysis: cytokines and calprotectin, antibody titers (ie. specific and non-specific IgE).

### Treatment of HIV Patient

An HIV-positive patient undergoing antiretroviral treatment is put on a diet rich in tryptophan, DHA, ARA, vitamin A and D and consumes 20 g of glycosylated whey protein/day along with 30 g of HMO in the form of 2′FL LNT LNnT, LNFP1, DFL and 6′SL. A cocktail of live bacteria including B. infantis, B. pseudocatulum, B. longum, and L. rhamnosus are each provided at 8 billion CFU/day given once per day. Enteric IFNβ is monitored routinely and if levels begin to drop, diet is modified to increase IFNβ levels.

### Example 6. Treatment of Hospitalized COVID Patient

A patient with COVID infection treated with steroids and/or nebulized IFN-B to slow the cytokine storm that is a hallmark of infection. Once stabilized the patient receives daily administration of 18 billion CFU of B. infantis EVC001. and an HMO water containing a 25 g/L solution of LNT:LNnT in a 10:1 ratio delivered via a nasogastric tube if in the ICU or orally if the patient can swallow. The patient is monitored for viral shedding, viral carriage, IFNβ and other cytokines enterically or systemically, as well as monitoring for recovery from clinical symptoms, or prevention of co-morbidities.

The age and type of disease or condition described in the examples are not intended to be all inclusive but rather demonstrate how compositions may be modified to suit a particular age and/or clinical need. The person skilled person is readily capable of making similar adjustments to the methods and compositions of this invention under comparable circumstances.

1. A method of elevating the level of interferon beta (IFNβ) in the gut of a mammal by providing said mammal with a live bacterium and a prebiotic that is consumed by said live bacterium.

2. The method of claim 1, wherein the mammal has or is at risk of one or more of the following conditions: an acute viral infection, a chronic viral infection, an autoimmune or allergic disease.

3. The method according to claim 1 for the treatment or prevention of gut dysbiosis associated autoimmune and allergic disease, said method comprising stimulating a therapeutically effective dose of interferon-beta to a mammal suffering from, or at risk of suffering from such disease.

4. The method of claim 3, wherein the method is intended to treat a mammal who has or is at risk of an autoimmune disease selected from the group consisting of rheumatoid arthritis, psoriasis, multiple sclerosis, celiac disease, inflammatory bowel diseases (Crohn's, ulcerative colitis,) IBS, Type 1 diabetes mellitus, and idiopathic pulmonary fibrosis.

5. The method of claim 3 wherein the method is intended to treat a mammal who has or is at risk of an allergic disease selected from the group consisting of atopic dermatitis, asthma, or food allergies.

6. The method of any one of claims 3-5, wherein a composition is used as an adjuvant or complementary treatment to drug therapy for a particular disease or condition.

7. A method of claim 6, wherein the adjuvant or complementary treatment increases the lifetime use of a drug or delays mammal becoming resistant to a drug commonly used for the treatment of such a condition.

8. The method of claim 5, wherein the method is intended to specifically prevent or treat food allergy.

9. The method of claim 8 wherein the method of treatment further comprises administering to the mammal desensitizing amounts of allergenic proteins. The method of claim 9, wherein the allergic protein is found in milk, egg, soy, wheat, shellfish, tree nut, peanut, fish, or sesame.

11. The method of claim 10, wherein the amount of allergenic protein is less than 1 mg, less than 5 mg, less than 10 mg, less than 20 mg, less than 30 mg, less than 40mg, less than mg, less than 60 mg, less than 100 mg or more than 100 mg.

12. The method of any one of claims 9-11, wherein the allergenic protein is added in increasing amounts over time as part of treatment regime.

13. The method according to claim 1 for the treatment or prevention of gut dysbiosis in mammals with acute or chronic viral infections comprising stimulating in said mammal a therapeutically effective dose of interferon-beta to a patient suffering from, or at risk of suffering from such infection.

14. The method of claim 13, where treatment intended to treat a mammal who has or is at risk of a virus causing a chronic viral infection, such as HIV, herpes, or hepatitis.

15. The method of claim 10 intended to treat a mammal who has or is at risk of an acute viral infection selected from a respiratory or gastrointestinal virus.

16. The method of claim 15 wherein the respiratory viruses may be selected from coronaviridae family, influenza, pneumonia, or respiratory syncytial virus.

17. The method of claim 16, wherein the virus is in the family Coronaviridae, optionally wherein the virus is SARS-CoV-2 (COVID19) or one of its variants.

18. A method of any one of claims 13-17, wherein treatment success may result in less viral shedding, viral carriage, less, complications, less neuronal complications, and/or improved recovery time.

19. The method of claim 1 for reducing viral carriage in a mammal by providing said mammal with an anti-viral composition comprising a live bacterium and a MMO that is consumed by that live bacterium.

20. The method of any of the preceding claims, wherein the composition contains one or more live bacterium selected from Bifidobacterium and/or Lactobacillus.

21. A method of any preceding claim wherein the live bacterium is Bifidobacterium selected from the group consisting of B. adolescentis, B. animalis, B. animalis subsp. animalis, B. animalis subsp. lactis, B. bifidum, B. breve, B. catenulatum, B. longum, B. longum subsp. infantis, B. longum subsp. longum, B. longum subsp. suis, B. pseudocatanulatum, and B. pseudolongum.

22. A method of claim 21, wherein the Bifidobacterium is B. infantis, optionally where it is B. infantis is strain EVC001 or a genetic equivalent to strain EVC001.

23. A method of claim 21, wherein the B. infantis has a functional H5 gene cluster and/or has function to cleave N-glycans from whey or other glycosylated proteins in vivo.

24. A method of any preceding claim wherein the live bacterium is Lactobacillus selected from the group consisting of L. acidophilus, L. brevis, L. casei, L. crispatus, L. curvatus, L. fermentum, L. pentosus, L. plantarum, and L. sakei L. antri, L. coleohominis, L. gasseri, L. johnsonii, L. mucosae, L. reuteri, L. rhamnosus, and L. salivarius.

25. A method of claim 24, wherein the Lactobacillus is L. rhamnosus or L. reuteri.

26. A method of claim 24, wherein L. rhamnosus is LGG.

27. A method of any of the preceding claims, wherein the live bacterium is activated and dried prior to administration.

28. A method of any of the preceding claims, wherein the live bacterium is stabilized and dried.

29. A method of claim 23, wherein the live bacterium is present 0.1 million to 500 billion colony forming units (CFU) per serving, per gram, per ml or per unit dose of the composition, optionally wherein the is present 1 million to 100 billion CFU or million to 50 billion CFU per serving, per gram, per ml or per unit dose of the composition.

30. A method of any one of claims 27-29, wherein the dried bacterium is suspended in oil.

31. A method of claim 30, wherein the oil is medium chain triglyceride (MCT), mineral, palm olein, soy, coconut, high oleic sunflower oil.

32. A method of any of the preceding claims wherein the prebiotic composition comprises a MMO, GOS, FOS, PDX or resistant starch.

33. A method of any preceding claim, wherein the prebiotic comprises one or more MMO selected from group consisting of lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof

34. A method of any preceding claim, wherein the prebiotic comprises MMO that is an HMO.

35. A method of claim 34, wherein the HMO is LNT and LNnT.

36. A method of claim 35, wherein LNT and LNnT are in 10:1 ratio or a ratio of 4:3.

37. A method of any preceding claim, wherein the prebiotic comprises MMO having at least one structure with sialic acid that is not sialyllactose, at least one structure with fucose that is not fucosyllactose, and at least one of LNT or LNnT.

38. A method of any one of claims 32-37, wherein the MMO comprises at least 2 MMO selected from 2′FL, 3′FL, 3′SL, 6′SL, LNT, LNnT, LNFP, and DFL.

39. A method of claim 38 further comprising GOS, FOS, PDX or resistant starch.

40. A method of claim 39, wherein the GOS is provided in a 1:10 to 10:1 ratio with the MMO.

41. A method of claim 40, wherein the GOS is preferably DP4 and DPS.

42. A method of any one of claims 32-40, wherein the prebiotic, MMO or HMO composition is provided in a sterile liquid, a gel or a paste.

43. A method of claim 42, wherein the sterile liquid is water with an HMO concentration of at least 1 g/L, at least 2 g/L, at least 4 g/L, at least 6 g/L, at least 8 g/L, at least 10 g/L, at least 12 g/L, at least 15 g/L, at least 20 g/L, or at least 30 g/L.

44. The method of claim 42 or 43, wherein the prebiotic in sterile liquid is consumed throughout the day to deliver a total daily intake of 1 gram (g), at least 4 g, at least 8 g, at least 12 g, at least 15 g, at least 20 g, at least 25 g, at least 30 g, at least 35 g, at least 40 g, at least 45 g, at least 50 g, or more than 50 g.

45. The method of any one of claims 42-44, wherein the prebiotic in sterile liquid is used to prepare infant formula or other prepared food for a mammal of any age.

46. The method of claim 45, wherein the infant formula does not contain FOS, GOS, PDX or other resistant starches.

47. The method of any of the preceding claims where in the mammal's diet is restricted to provide low or no non-MMO dietary fiber.

48. The method of any preceding claim wherein the live bacterium is administered once daily.

49. The method of any preceding claim wherein the daily intake of MMO is modified to increase or decrease based on effectiveness of elevating IFNβ.

50. The method of any preceding claim wherein the live bacterium and HMO are provided contemporaneously in a stabilized powder format.

51. The method of any of the preceding claims wherein the composition further comprises DHA and ARA.

52. The method of any one of the preceding claims, wherein the composition further comprises vitamin A and/or vitamin D.

53. The method of any one of the preceding claims, wherein the composition further comprises glycosylated whey protein and/or tryptophan or a milk fat globule membrane complex.

54. The method of treatment for any preceding claim further comprising pre-administration and/or contemporaneous administration of an exogenous source of IFNβ to a mammal, said administration may be oral, rectal, intramuscular, intravenous, or in a nebulized form.

55. The method of any one of the preceding claims, wherein the composition is administered as a unit dose, a medicament, a food, an infant food, a medical food, a dietary supplement, or a pharmaceutical agent.

56. The method of any one of the preceding claims, wherein the composition is delivered as part of a dietary regime specific to the needs of the mammal during treatment.

57. The method of claim 55, wherein the food is an infant food, or a food for a mammal with an acute viral infection, a chronic viral infection, an autoimmune or allergic disease.

58. The method of any preceding claims, wherein composition is used to supplement human milk, human milk fortifier of human or bovine origin, infant formula, follow-on formula, a prepared food, enteral nutrition, or a meal replacer for a subject of any age.

59. The method of any one of the preceding claims, wherein said mammal is a human, non-human primate, cow, horse, sheep, pig, goat, buffalo, dog or cat.

60. The method of claim 59, wherein the human mammal is selected from an age group comprising infant 0-3 months, infant 3-6 months, infant 6-12 months, toddler 1-3 years, child 3-5, 5-10, 1-16, 16-18 years or an adult 18+, 20+, 30+, 40+, 50+, 60+, 70+ 80+, 90+ or 100+ years.

61. The method of any one of the preceding claims wherein the IFNβ is measured enterically or systemically from a stool or blood sample.

62. The method of claim 61, wherein the measurement from said stool or blood sample is used to diagnose need and/or monitor effectiveness of compositions used in treatment.

63. The method of claim 61 or 62 for maintaining IFNβ levels, further comprising monitoring Bifidobacteriaceae, and/or certain cytokines, calprotectin, Enterobacteriaceae to tailor treatment to achieve necessary clinical improvements.

64. A method to improve symptoms of rheumatoid arthritis or psoriasis, said method comprising delivering a live bacterium and MMO to a mammal having said symptoms, and monitoring increase in IFNβ and decrease IL-7, IL-21 and IL-22.

65. The method of claim 61 or 62 for monitoring effectiveness of treatment for food allergy and other allergic diseases comprising monitoring clinical symptoms and/or monitoring increased IFNβ production and/or decreased IL-4, IL-5 and IL-13, and/or antigen specific IgE reactions enterically and/or systemically.

66. The method of claim 61 or 62 for maintaining IFNβ production, further comprising monitoring enteric and systemic inflammation to prevent HIV-1 progression of disease towards AIDS.

67. A method for reducing viral carriage in a mammal by providing said mammal with an anti-viral composition comprising a live bacterium and a MMO that is consumed by that live bacterium.

68. The method of claim 67, wherein reducing the carriage of a virus in a human comprises the steps of:
a. providing the patient that is infected with the virus with from 10⁶ to 10¹¹ cfu of B. infantis contemporaneously with from 1-50 g of a mammalian milk oligosaccharide;
b. monitoring the patient for the presence of the virus; and
c. repeating the daily dosing until the titer of the virus drops by 95%.

69. The method for reducing the carriage of a Corona Virus in a human, said method comprising the steps of:
a. providing the patient that is infected with a Corona Virus with from 10⁶ to 10¹¹ cfu of B. infantis contemporaneously with from 1-50 g of a mammalian milk oligosaccharide;
b. monitoring the patient for the presence of a Corona Virus; and
c. repeating the daily dosing until the titer of the Corona Virus drops by 95%.

70. A method for reducing the severity of infection and/or comorbidities of a human with a Corona Virus, said method comprising the steps of:
a. providing the individual with from 10⁶ to 10¹¹ cfu of B. infantis contemporaneously with from 1-50 g of a mammalian milk oligosaccharide; and
b. repeating the daily dosing until through the time of a viral outbreak.

71. A method for preventing infection of a human who may have been in contact with another person who was carrying a Corona Virus, said method comprising the steps of:
a. providing the human with from 10⁶ to 10¹¹ cfu of B. infantis contemporaneously with from 1-50 g of a mammalian milk oligosaccharide;
b. monitoring the human for the presence of a Corona Virus; and
c. repeating the daily dosing for from 14-21 days.
